(19) United States
(12) Patent Application Publication
(10) Pub. No.: US 2006/0089888 A1
(43) Pub. Date: Apr. 27, 2006

(54) Medical implant distribution method and system

(76) Inventor: Gregory James Roger, New South Wales (AU)

Correspondence Address: JACOBSON HOLMAN PLLC, 400 SEVENTH STREET N.W., SUITE 600, WASHINGTON, DC, 20004, US

(21) Appl. No.: 11/252,887

(22) Filed: Oct. 19, 2005

(30) Foreign Application Priority Data
Oct. 19, 2004 (AU) 2004906053

(51) Int. Cl. G06Q 20/00; G06G 1/14

(52) U.S. Cl. 705/28; 705/22

(57) ABSTRACT

A method of supply of medical implants directly from an implant manufacturer to a hospital without going via a distributor, allows the manufacturer to sell an implant to the hospital at the same price as they would ordinarily sell to a distributor which provides the hospital with the implant at a much reduced cost. The hospital may use its own in-house management and storage abilities to maintain an inventory of implants resulting in increased efficiencies and cost savings. Hence, when a surgeon at a hospital wishes to obtain, say, a knee implant for an operation, instead of having to go to a distributor for the implant, the surgeon can simply obtain the implant from the hospital's own inventory management system. Preferably, the inventory management system incorporates an inventory based management system which interfaces with the hospital's stock management system and that of the manufacturer.

### CROSS-REFERENCE TO RELATED APPLICATIONS

The present application claims priority from Australian Provisional Patent Application No 2004906053 filed on 19 Oct. 2004, the entire contents of which are incorporated herein by reference.

### FIELD OF THE INVENTION

This invention relates to a medical implant distribution system.

### BACKGROUND OF THE INVENTION

Currently the manufacturing, distribution and sales model for medical implants (prostheses) such as artificial knees, hips and the like is for a manufacturer of such implants to make and stock a certain number of implants. A distributor purchases some of the stock and has a certain territory in which they try and sell the implants to the end user—typically hospitals. In order to do this they need promotional material, sales people and stock management infrastructure. Distributors also require offices, warehouses and management. Generally speaking, when a medical implant is sold, 50 percent of the purchase price will go to the manufacturer and approximately 50 percent to the distributor to cover the costs of the distribution and sales channels and provide a profit for the distributor.

Medical implants are implanted in patients in hospitals. For most implant operations, a health fund will reimburse the hospital a lump sum to cover all costs of the implant operation including the prothesis. Margins on hospital implant operations in the USA are low. Typically, a hospital in the United States of America might make a profit of around US$400 per total knee implant which works out at about a 2% return on a typical operation, which is not a good margin. Any reduction in the purchase price of the implant improves the hospital's operating margins.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

In a first broad aspect of the present invention the method of the present invention involves the supply of medical implants directly by a manufacturers to a hospital without going via a distributor. This allows the manufacturer to sell an implant to the hospital at the same price as they would ordinarily sell to a distributor which provides the hospital with the implant at a much reduced cost.

In a related feature of the invention the hospital may use its own in house management and storage abilities to maintain an inventory of implants.

Most hospitals already have storage inventory management and ordering management capabilities for their general equipment and consumables such as pharmaceuticals. The hospital uses its own in store management and storage abilities instead of the distributor's resulting in increased efficiencies and cost savings.

Hence, when a surgeon at a hospital wishes to obtain say a knee implant for an operation, instead of having to go to a distributor for the implant, the surgeon can simply obtain the implant from the hospital's own inventory management system.

Preferably, the inventory management system for implants interfaces with the hospital's stock management system and that of the manufacturer.

In order to implement the system the hospital typically require an extra health worker, such as a nurse who is required to have knowledge of the implants and implant techniques. Typically the manufacturer would supply free training to the nurse. The hospital purchases the inventory they require and store and manage the stock, and reorder as required.

The inventory management system could be maintained by the manufacturer on the manufacturer's computer system and accessed by the hospital via the internet.

The manufacturer may also maintain recommended inventory levels for hospitals and lists of components required and instruments required for particular operations which can be accessed by the hospital's inventory management system to ensure that their inventory levels are appropriate.

The inventory management system may be capable of receiving information in advance about planned operations and automatically adjusting inventory levels in advance in the expectation that those operations being carried out to maintain an appropriate inventory at the hospital.

The process of re-ordering implants may be adapted to assist in the tracking of implants. In particular the process of the hospital registering the use of an implant and triggering a re-order from the manufacturer could be adapted to also make a record of the product lot/serial number and patient information for retention by the hospital.

In this way the hospital may generate a computer based registry system maintaining a record of product serial number and patient details. This would allow the hospital to more efficiently effect a recall if required due to e.g. defects in the implant. The tracking information could also be made available to a national register of implants.

Currently hospitals store implant details in a patients notes which are not easily searchable.

### BRIEF DESCRIPTION OF THE DRAWINGS

A specific example of the present invention will now be described by way of example only with reference to the accompanying drawings in which—:

FIG. 1 is a schematic illustration of a hospital based inventory management system linked via the internet to a manufacturer's computer ordering and shipment system.

### DETAILED DESCRIPTION OF A PREFERRED EMBODIMENT

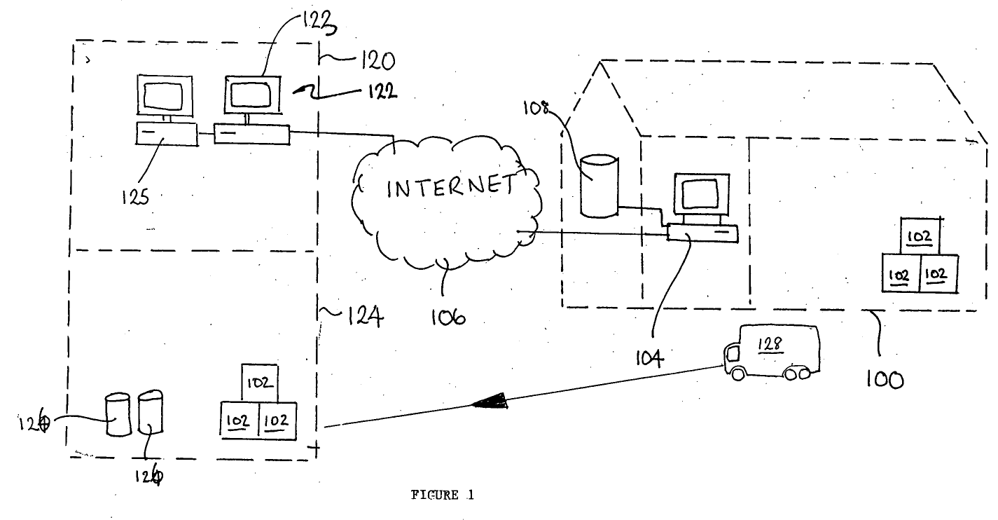

Referring to the drawings, FIG. 1 shows a factory 100 manufacturing surgical implants 102. Such medical implants may include, for example, artificial knees, artificial hips, artificial shoulders or the like. Typically they will be used to replace a patient's joint which has been damaged or, more typically, has become worn out. The factory includes a computer based sales system 104 for receiving, processing, and shipping sales orders for surgical implants 102 and maintains its own inventory of products for sale including implants 102. The sales system 104 may be linked to the internet 106 by a modem or broadband link or the like to receive sales orders. Sales orders may also be placed at the factory by other methods such as telephone, ordinary mail or the like. The computer sales system 104 may be linked to a database 108.

FIG. 1 also shows a schematic diagram of a hospital 120 including a computer based storage inventory management and ordering system 122. The system includes a computer means 123 having an internal memory or database, or optionally an external database, for maintaining a record of physical product inventory. The system may include a means for automatically ordering a replacement product from suppliers when inventories are low embodied in software running on the computer 123. This may be effected by generating printed orders or may be effected automatically through electronic means via the internet or electronic transmission network means. The system will typically manage procurement of most, if not all, hospital supplies particularly pharmaceuticals, dressings and other consumables, and in the present invention will also store and maintain an inventory record for medical implants and related components and instruments associated with operations to install such implants. However, it would be possible to have a stand alone system shown at 125 just for implants, preferably linked to the system 123. Typically the implants 102 will be stored in a secure storage room 124 for pharmaceuticals 126 and other high value consumables. The implants may be despatched directly to the hospital 120 from the factory 100 by truck 128 or any other suitable transport means.

The system 122 may hold information about the use of the implants such as the instruments and associated inventory required for the operation to implant the prothesis. This may also be available on the factory database 108 and made available to approved consumers via the internet.

However, it is desirable that at least one person at the hospital is trained in the knowledge of instruments and techniques of what inventory is required for particular implant operations.

The hospital 120 purchases their own inventory directly from the manufacturer and store and manage their own stock, and reorder as required.

A typical knee implant purchased directly from the manufacturer might cost US$2000.00 as compared with US$4000 via a distributor The net cost of storage and management of inventory might amount to approximately 40% of the sale price from the manufacture i.e. about US$800 This results in a cost saving of US$1200 goes straight to the hospitals bottom line.

Typically, a hospital in the United States of America might make a profit of around US$400 per total knee implant which works out at about 2% return on a typical operation which is not a good margin. The cost saving of approximately US$1200 increases the hospitals profit to around $1,600 per operation and an 8% profit margin which is four times the typical existing profit margin.

In an alternative embodiment the inventory management system could be maintained by the manufacturer on their computer and database system 104, 108 and accessed by the hospital via the internet. The manufacturer may also maintain recommended inventory levels or hospitals and lists of components required and instruments required for particular operations which can be accessed by the hospital inventory management system to ensure that their inventory levels are appropriate.

The inventory management system may be capable of receiving information in advance about planned operations and automatically adjusting inventory levels in advance in the expectation that those operations being carried out and to maintain an appropriate inventory at the hospital.

The process of re-ordering implants may be adapted to assist in the tracking of implants. In particular the process of the hospital registering the use of an implant and triggering a re-order from the manufacturer could be adapted to also make a record of the product lot/serial number and patient information for retention by the hospital. This record may be maintained electronically on the hospital's computer system 123 or 125 or even on a separate linked database (not shown). The registry may be searchable by implant type and serial number.

In this way the hospital may generate a computer based registry system maintaining a record of product serial number and patient details. This would allow the hospital to more efficiently effect a recall if required due to e.g. defects in the implant. The tracking information could also be made available to a national register of implants.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of supplying medical implants from a manufacturer of medical implants to a hospital where a patient may undergo an operation to receive such a medical implant, wherein the medical implants are despatched directly to the hospital from the manufacturer rather than via a distributor, and including the hospital maintaining its own inventory of medical implants, including a stock of medical implants and an inventory record, for implantation into patients.

2. A method as claimed in claim 1 wherein the medical implants are selected from the group including artificial knees, artificial shoulders, and artificial hips.

3. A method as claimed in claim 1 wherein the hospital includes a stock management and storage system for maintaining an inventory of pharmaceuticals, wound dressings and other consumables and further including maintaining the inventory record of medical implants on said stock management and storage system.

4. A method as claimed in claim 1 wherein the hospital includes a stock management and storage system for maintaining an inventory record of pharmaceuticals, wound dressings and other consumables and wherein a separate medical implants inventory management system is maintained at the hospital to maintain a record of the hospital's inventory of medical implants.

5. A method as claimed in claim 4 wherein the separate medical products inventory management system interfaces with the hospital's stock management and storage system and wherein the manufacturer maintains a computer based system for receiving processing and shipping orders with which the separate medical products inventory management system also interfaces.

6. A method as claimed in claim 1 including the step of the manufacturer supplying training in implants and implant techniques to one or more operatives at the hospital.

7. A method as claimed in claim 1 wherein the hospital's inventory record of medical implants is maintained by the manufacturer on a computer system and wherein the inventory record is accessible by the hospital via the internet.

8. A method as claimed in claim 1 wherein the manufacturer maintains information regarding recommended inventory levels of artificial implants for hospitals and lists of components required and instruments required for conducting implant operations which can be accessed by the hospital's inventory management system.

9. A method as claimed in claim 1 wherein information regarding planned operations is supplied in advance to the inventory record of medical products and wherein inventory levels are adjusted in advance in the expectation of those operations being carried out to maintain an appropriate medical implants stock inventory at the hospital.

10. A method as claimed in claim 9 wherein when the hospital registers use of an implant and triggers a re-order from the manufacturer a record of the implant details including its serial or lot number and patient information is caused to be made in an electronic register maintained at the hospital.

11. A method of supplying medical implants including artificial knees from a manufacturer of medical implants to a hospital where a patient may undergo an operation to receive such a medical implant, the hospital including a stock management and storage system for maintaining an inventory of pharmaceuticals, wound dressings and other consumables and including the step of maintaining an inventory record of medical implants at the hospital on said stock management and storage system, and a physical stock of medical implants at the hospital, and including the step of despatching medical implants directly to the hospital from the manufacturer rather than via a distributor.

12. A method as claimed in claim 11 wherein the medical implants are selected from the group including artificial knees, artificial shoulders, and artificial hips.

12. A method as claimed in claim 11 including the step of the manufacturer supplying training in implants and implant techniques to one or more operatives at the hospital.

13. A method as claimed in claim 12 wherein the manufacturer maintains information regarding recommended inventory levels for hospitals and lists of components required and instruments required for conducting implant operations which can be accessed by authorised operative at the hospital.

14. A method as claimed in claim 12 wherein when the hospital registers use of an implant and triggers a re-order from the manufacturer a record of the implant details including its serial or lot number and patient information is made in an electronic register maintained on said stock management system or on a computer means or database linked to said system.
